(19) United States
(12) Patent Application Publication
(10) Pub. No.: US 2026/0038678 A1
(43) Pub. Date: Feb. 5, 2026

(54) COMPUTATIONAL ARCHITECTURE FOR REMOTE IMAGING EXAMINATION MONITORING TO PROVIDE ACCURATE, ROBUST AND REAL-TIME EVENTS

(71) Applicant: KONINKLIJKE PHILIPS N.V., EINDHOVEN (NL)

(72) Inventors: SIVA CHAITANYA CHADUVULA, MALDEN, MA (US); OLGA STAROBINETS, NEWTON, MA (US); RANJITH NAVEEN TELLIS, TEWKSBURY, MA (US); EKIN KOKER, CAMBRIDGE, MA (US); SANDEEP MADHUKAR DALAL, WINCHESTER, MA (US); THOMAS ERIK AMTHOR, HAMBURG (DE); YUECHEN QIAN, LEXINGTON, MA (US); PHILIP OXENBERG, CAMBRIDGE, MA (US)

(21) Appl. No.: 18/997,291

(22) PCT Filed: Jul. 26, 2023
(86) PCT No.: PCT/EP2023/070681

§ 371 Date: Jan. 21, 2025

Related U.S. Application Data
(60) Provisional application No. 63/392,550, filed on Jul. 27, 2022.

(30) Foreign Application Priority Data
Aug. 31, 2022 (EP) 22193278.3

(51) Int. Cl. G16H 40/63

(52) U.S. Cl. CPC G16H 40/63

(57) ABSTRACT

A method (100) of monitoring a medical imaging examination is described. The method includes receiving one or more video feeds (17) of at least an imaging bay (3); detecting, from the one or more video feeds, whether a medical procedure is being performed in the imaging bay; in response to the detecting indicating a medical procedure is being performed in the imaging bay, controlling a local electronic processing device (8) assigned to the imaging bay to process the one or more video feeds to extract and present information about the medical procedure being performed in the imaging bay; and in response to the detecting indicating a medical procedure is not being performed in the imaging bay, controlling the local electronic processing device to not process the one or more video feeds.

### FIELD OF THE INVENTION

The following relates generally to the imaging arts, remote imaging assistance arts, remote imaging examination monitoring arts, and related arts.

### BACKGROUND OF THE INVENTION

Medical imaging, such as computed tomography (CT) imaging, magnetic resonance imaging (MRI), positron emission tomography (PET) imaging, fluoroscopy imaging, and so forth, is a critical component of providing many types of medical care, and is used in a wide range of medical fields, such as cardiology, oncology, neurology, orthopedics, to name a few. The operator of the medical imaging device used to acquire the medical images is typically a trained technologist, while interpretation of the medical images is often handled by a medical specialist such as a radiologist. Interpretation of radiology reports or findings by the radiologist, and application of those findings to the patient's specific clinical case, can be handled by the patient's general practitioner (GP) physician or a medical specialist such as a cardiologist, oncologist, orthopedic surgeon, or so forth.

Currently, diagnostic imaging is in high demand. As the world population ages, the demand for quick, safe, high quality imaging will only continue to grow, putting further pressure on imaging centers and their staff. One approach for imaging centers to boost efficiency and grow operations without concomitant increase in labor costs is through a radiology operations command center (ROCC) system. Radiology operations command centers enable teams to work across the entire network of imaging sites, providing their expertise as needed and remotely assisting less experienced technologists in carrying out high quality scans. Remote technologists or experts can monitor the local operators of scanning procedures through cameras installed in the scanning areas or from other sources, such as sensors (including radar sensors), console video feeds, microphones connected to Internet of Things (IoT) devices, and so forth. In addition, these sources can be supplemented by other data sources like Health-Level 7 (HL7) medical data feeds, medical images stored in Digital Imaging and Communications in Medicine (DICOM) format, Electronic Health Record (EHR) databases, and so forth.

ROCC enables telepresence via audio-video connectivity and provides real-time access to imaging scanner console screens and video camera feeds from scanner rooms to a remote command center. The expert users at the command center provide virtual over-the-shoulder support to the local technologists and staff conducting imaging exams. The ROCC can also provide automated assistance to the remote expert and/or local technologist. Computational algorithms on ROCC tablet hardware process real-time event data from multiple channels including console and camera and provide actionable insights to the expert technologist in real time. ROCC offers solutions for a wide range of scanning devices such as MRI, CT, X-ray, and Ultrasound and the duration of patient in a scanner room varies from a few minutes to few hours. It is important that the computations run within ROCC are run in way that these events are reliably generated in real time across different imaging modalities. Such capabilities enable expert technologists to intervene proactively based on the events and support the local technologists in their day-to-day issues.

The following discloses certain improvements to overcome these problems and others.

### SUMMARY OF THE INVENTION

The invention is defined by the independent claims. Dependent claims represent beneficial embodiments.

In one aspect, a non-transitory computer readable medium stores instructions executable by at least one electronic processing device to perform a method of monitoring a medical imaging examination. The method includes receiving one or more video feeds of at least an imaging bay; detecting, from the one or more video feeds, whether a medical procedure is being performed in the imaging bay: in response to the detecting indicating a medical procedure is being performed in the imaging bay, controlling a local electronic processing device assigned to the imaging bay to process the one or more video feeds to extract and present information about the medical procedure being performed in the imaging bay; and in response to the detecting indicating a medical procedure is not being performed in the imaging bay, controlling the local electronic processing device to not process the one or more video feeds.

In another aspect a method of monitoring a medical imaging examination is described, the method comprising the steps of:
receiving one or more video feeds of at least an imaging bay;
detecting, from the one or more video feeds, whether a medical procedure is being performed in the imaging bay;
in response to the detecting indicating a medical procedure is being performed in the imaging bay, controlling a local electronic processing device assigned to the imaging bay to process the one or more video feeds to extract and present information about the medical procedure being performed in the imaging bay; and
in response to the detecting indicating a medical procedure is not being performed in the imaging bay, controlling the local electronic processing device to not process the one or more video feeds.

It is to be understood that method and computer-readable medium for performing the method might be considered as synonyms, and if the applicant states computer-readable medium, that might refer to a method, and vice-versa, when the applicant states a method, that might refer to a computer-readable medium for performing the method.

In another aspect, a support apparatus for medical imaging includes a server computer; and local electronic processing devices assigned to respective medical imaging bays and programmed to apply machine learning (ML) models to video feeds received from their respective assigned imaging bays to extract information about medical imaging procedures performed in their respective assigned imaging bays. The server computer and/or the local electronic processing devices are programmed to determine whether medical imaging procedures are being performed in the respective medical imaging bays. The server computer is programmed to perform training of the ML models including allocating ML model training tasks amongst the local electronic processing devices based on whether medical imaging procedures are being performed in the corresponding assigned medical imaging bays and receiving results of the allocated ML model training tasks from the local electronic processing devices.

In another aspect, a non-transitory computer readable medium stores instructions executable by at least one electronic processing device to perform a method of monitoring a medical imaging examination. The method includes receiving one or more video feeds of at least an imaging bay; detecting, from the one or more video feeds, whether a medical procedure is being performed in the imaging bay: in response to the detecting indicating a medical procedure is being performed in the imaging bay, controlling a local electronic processing device assigned to the imaging bay to process the one or more video feeds to extract and present information about the medical procedure being performed in the imaging bay; and in response to the detecting indicating a medical procedure is not being performed in the imaging bay, controlling the local electronic processing device to perform one or more training tasks for a machine learning (ML) component.

A computer program comprising instructions which, when the program is executed by a computer, cause the computer to carry out the steps of any of the embodiments of the present invention is further described.

One advantage resides in processing images during a medical imaging examination more quickly.

Another advantage resides in conserving computing power when medical imaging examinations are not being performed.

Another advantage resides in training machine-learning (ML) models to perform imaging analysis tasks.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 shows an example flow charts of operations suitably performed by the apparatus of FIG. 1.

FIGS. 3 and 4 show different embodiments of the apparatus of FIG. 1.

FIG. 5 shows an example flow charts of operations suitably performed by the apparatus of FIG. 1.

### DETAILED DESCRIPTION OF EMBODIMENTS

As earlier discussed, the ROCC can provide automated assistance to the remote expert and/or local technologist. Such automated assistance may be provided in the form of machine learning (ML)-based image analysis performed on medical images immediately upon their acquisition. Such ML-based analyses can provide early detection of imaging artifacts, poorly chosen field-of-view, incidental findings beyond the reason for the imaging examination, and other issues. By way of early detection of such issues, additional or replacement images can be acquired during the imaging examination, thereby eliminating the need for a call-back examination. ML-based detection of incidental findings can also have immense clinical value by providing early detection of treatable medical conditions. The ROCC may utilize other types of ML models, for example ML models applied to bay camera video to detect events occurring in the imaging bay.

However, the ML models used in such ML-based image analysis require training, which is a computationally complex process. A large suite of ML models can be envisioned to provide detection of a wide range of different types of artifacts, non-optimal imaging conditions, incidental findings, and so forth. However, the training of such a suite of ML models can be taxing even for a server computer or network of servers. Moreover, it may be desirable for some types of ML models to be dynamically trained, i.e. to have update training on a regular basis using recently acquired medical images, in order to ensure the ML models are well-tuned for the particularities of images acquired by newer models of medical imaging devices and newer techniques in medical imaging such a use of new types of contrast agent, new imaging sequences, or so forth.

The following discloses leveraging a layered information technology (IT) architecture to better utilize available computing resources and improve ML-based analyses of complex image and audio analyses to extract actionable information from the bay camera video and possibly other video cameras (in-bore, contrast injector, et cetera) and from a microphone in the imaging bay. Such ML analyses are computationally taxing, and can be prone to error if the training dataset is insufficient.

The layered IT architecture includes the sensors, the “edge” devices, the cloud computing layer, and a centralized application layer. The “edge” devices may, for example, be tablet computers or the like used by the local imaging technician to interface with the centralized application layer. The cloud computing layer corresponds, for example, to the hospital's computing IT network, while the centralized application layer is maintained by a vendor of a medical imaging device. In this architecture, the edge devices carry a substantial workload including camera feed analyses, imaging scanner console scraped screen acquisition and analyses, microphone audio analysis, and conversion to actionable events using various ML models. This is problematic since the edge devices (e.g. tablet computers) typically have limited resources such as computational capacity. On the other hand, while the edge devices have limited computational capacity, they are numerous in a large ROCC network, and as recognized herein they can be effectively leveraged in the aggregate to assist in complex computational tasks.

In one aspect, a training orchestrator tracks usage of the edge devices and reallocates the computing power of those devices that are experiencing downtime (e.g. between examinations, during night shifts, et cetera) to ML model training tasks. The orchestrator also coordinates the ML model training tasks, for example storing an index of ML models and allocating training to edge devices utilizing those models, and tracking success/failure ratios of the ML models to allocate or prioritize which models to train. This aspect leverages the edge devices in the aggregate for the secondary task of assisting in ML model training, without adversely impacting their primary task of assisting imaging technicians and/or experts in imaging examinations.

In another aspect, optical character recognition (OCR) and information extraction from the console display feed are divided into mandatory and conditional categories. Mandatory OCR tasks are those that relate to fields that can in general change throughout the imaging examination, so that the OCR needs to be run throughout the scan. Conditional OCR tasks are those that relate to fields that only change at well-defined phases of the examination. An example of conditional OCR tasks are those relating to patient demographics, which is entered only during the exam startup phase and thereafter remain unchanged Conditional OCR tasks are only performed during their relevant exam phase(s), thus reducing computational load on the edge device. This aspect increases the efficiency of the edge devices in performing their primary task of assisting imaging technicians and/or experts in imaging examinations, by removing the unnecessary processing load entailed in OCR'ing text that is static over a significant portion of an imaging examination.

In another aspect, rather than applying a single ML model to extract information, an ensemble of ML models can be applied, and the final result obtained by combining the outputs of the ensemble using a technique such as voting. This aspect improves the effectiveness of the ML models in assisting imaging technicians and/or experts in imaging examinations.

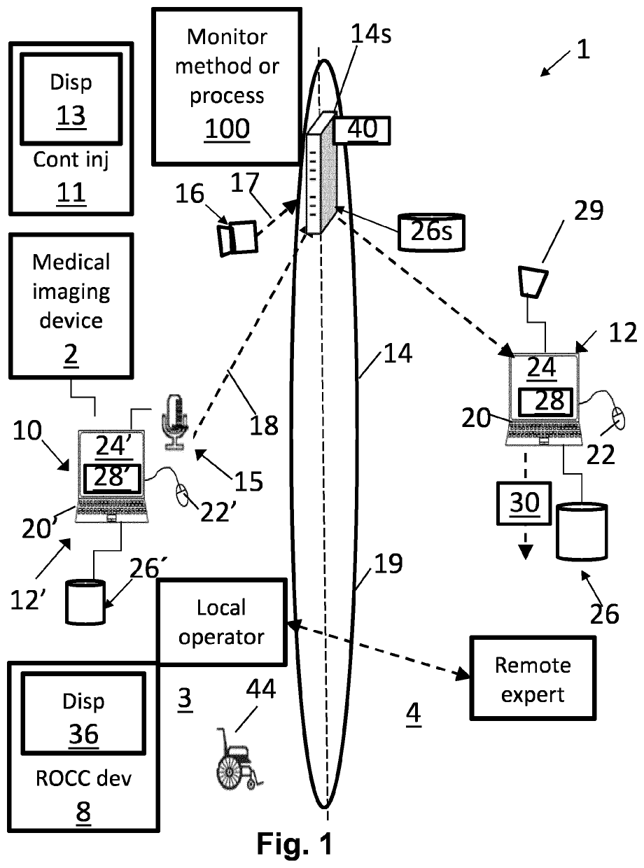

With reference to FIG. 1, a support apparatus 1 for providing assistance from a remote medical imaging expert RE (or supertech) to a local technologist operator LO is shown. As shown in FIG. 1, the local operator LO, who operates a medical imaging device (also referred to as an image acquisition device, imaging device, and so forth) 2, is located in a medical imaging device bay 3, and the remote expert RE is disposed in a remote service location or center 4. It should be noted that the “remote expert” RE may not necessarily directly operate the medical imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the medical imaging device bay 3 (this may, for example, in the case of a “remote operator or expert” RE who is a radiologist tasked with peri-examination image review), but more typically the remote service center 4 and the medical imaging device bay 3 are in different buildings, and indeed may be located in different cities, different countries, and/or different continents. In general, the remote location 4 is remote from the imaging device bay 3 in the sense that the remote expert RE cannot directly visually observe the imaging device 2 in the imaging device bay 3 (hence optionally providing a video feed as described further herein).

The image acquisition device 2 can be a Magnetic Resonance (MR) image acquisition device, a Computed Tomography (CT) image acquisition device: a positron emission tomography (PET) image acquisition device: a single photon emission computed tomography (SPECT) image acquisition device: an X-ray image acquisition device: an ultrasound (US) image acquisition device: or a medical imaging device of another modality. The imaging device 2 may also be a hybrid imaging device such as a PET/CT or SPECT/CT imaging system. While a single image acquisition device 2 is diagrammatically represented in FIG. 1, more typically a medical imaging laboratory will have multiple image acquisition devices, which may be of the same and/or different imaging modalities. For example, if a hospital performs many CT imaging examinations and relatively fewer MRI examinations and still fewer PET examinations, then the hospital's imaging laboratory (sometimes called the “radiology lab” or some other similar nomenclature) may have three CT scanners, two MRI scanners, and only a single PET scanner. This is merely an example. Moreover, the remote service center 4 may provide service to multiple hospitals. The local operator controls the medical imaging device 2 via an imaging device controller 10. The remote operator is stationed at a remote workstation 12 (or, more generally, an electronic controller 12).

Some types of imaging modalities may utilize an intravascular contrast agent. For example, MR may utilize a gadolinium-based contrast agent. To provide for contrast-enhanced imaging, a contrast injector 11 is configured to inject the patient with a contrast agent. The contrast injector 11 is a configurable automated contrast injector having a display 13. The user (usually the imaging technologist) loads a vial or syringe of contrast agent (or two, or more, vials of different contrast agent components) into the contrast injector 11, and configures the contrast injector 11 by entering contrast injector settings such as flow rates, volumes, time delays, injection time durations, and/or so forth via a user interface (UI) of the contrast injector 11. The UI may be a touch-sensitive overlay of the display 13, and/or physical buttons, keypad, and/or so forth. In a variant embodiment, the contrast injector 11 is integrated with the imaging device controller 10 (e.g., via a wired or wireless data connection), and the contrast injector 11 is controlled via the imaging device controller 10, including displaying the contrast injector settings in a (optionally selectable) window on the display of the imaging device controller 10. In such an embodiment, the dedicated physical injector display 13 of the contrast injector may optionally be omitted (or, alternatively, the dedicated physical injector display 13 may be retained and the contrast settings displayed at both the dedicated physical injector display 13 and at the imaging device controller 10). In general, the automated contrast injector 11 can employ any suitable mechanical configuration for delivery of the contrast agent (or agents), such as being a syringe injector, a dual-syringe injector, pump-driven injector, or so forth, and may include hardware for performing advanced functions such as saline dilution of the contrast agent, priming and/or flushing of the contrast injection line with saline, and/or so forth.

As used herein, the term “medical imaging device bay” (and variants thereof) refer to a room containing the medical imaging device 2 and also any adjacent control room containing the medical imaging device controller 10 for controlling the medical imaging device. For example, in reference to an MRI device, the medical imaging device bay 3 can include the radiofrequency (RF) shielded room containing the MRI device 2, as well as an adjacent control room housing the medical imaging device controller 10, as understood in the art of MRI devices and procedures. On the other hand, for other imaging modalities such as CT, the imaging device controller 10 may be located in the same room as the imaging device 2, so that there is no adjacent control room and the medical bay 3 is only the room containing the medical imaging device 2. In addition, while FIG. 1 shows a single medical imaging device bay 3, it will be appreciated that the remote service center 4 (and more particularly the remote workstation 12) is in communication with multiple medical bays via a communication link 14, which typically comprises the Internet augmented by local area networks at the remote expert RE and local operator LO ends for electronic data communications. In addition, while FIG. 1 shows a single remote service center 4, it will be appreciated that the medical imaging device bays 3 is in communication with multiple medical bays via the communication link 14.

As diagrammatically shown in FIG. 1, in some embodiments, a camera 16 (e.g., a video camera) is arranged to acquire a video stream or feed 17 of a portion of a workspace of the medical imaging device bay 3 that includes at least the area of the imaging device 2 where the local operator LO interacts with the patient, and optionally may further include the imaging device controller 10. While one camera 16 is shown, there may be multiple cameras. e.g. one providing a feed of the imaging bay generally, another providing a video feed of a display 36 of the contrast injector 11, and/or so forth. In other embodiments, a microphone 15 is arranged to acquire an audio stream or feed 18 of the workspace that includes audio noises occurring within the medical imaging device bay 3 (e.g., verbal instructions by the local operator LO, questions from the patient, and so forth). The video stream 17 and/or the audio stream 18 is sent to the remote workstation 12 via the communication link 14. e.g. as a streaming video feed received via a secure Internet link.

The communication link 14 also provides a natural language communication pathway 19 for verbal and/or textual communication between the local operator and the remote operator. For example, the natural language communication link 19 may be a Voice-Over-Internet-Protocol (VOIP) telephonic connection, an online video chat link, a computerized instant messaging service, or so forth. Alternatively, the natural language communication pathway 19 may be provided by a dedicated communication link that is separate from the communication link 14 providing the data communications 17, 18, e.g. the natural language communication pathway 19 may be provided via a landline telephone. In some embodiments, the natural language communication link 19 allows a local operator LO to call a selected remote expert RE. The call, as used herein, can refer to an audio call (e.g., a telephone call), a video call (e.g., a Skype or Facetime or other screen-sharing program), or an audio-video call. In another example, the natural language communication pathway 19 may be provided via an ROCC device 8, such as a mobile device (e.g., a tablet computer or a smartphone), or can be a wearable device worn by the local operator LO, such as an augmented reality (AR) display device (e.g., AR goggles), a projector device, a heads-up display (HUD) device, etc., each of which having a display device 36. For example, an “app” can run on the ROCC device 8 (operable by the local operator LO) and the remote workstation 12 (operable by the remote expert RE) to allow communication (e.g., audio chats, video chats, and so forth) between the local operator and the remote expert. In some examples, when multiple imaging device bays 3 can each include a corresponding ROCC device 8.

FIG. 1 also shows, in the remote service center 4 including the remote workstation 12, such as an electronic processing device, a workstation computer, or more generally a computer, which is operatively connected to receive and present the video feed 17 of the medical imaging device bay 3 from the camera 16 and/or to the audio feed 18. Additionally or alternatively, the remote workstation 12 can be embodied as a server computer or a plurality of server computers, e.g. interconnected to form a server cluster, cloud computing resource, or so forth. The workstation 12 includes typical components, such as an electronic processor 20 (e.g., a microprocessor), at least one user input device (e.g., a mouse, a keyboard, a trackball, and/or the like) 22, and at least one display device 24 (e.g. an LCD display, plasma display, cathode ray tube display, and/or so forth). In some embodiments, the display device 24 can be a separate component from the workstation 12. The display device 24 may also comprise two or more display devices. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24. The video feed 17 from the camera 16 can also be displayed on the display device 24, and the audio feed 18 can be output on the remote workstation 12 via a loudspeaker 29. In some examples, the audio feed 18 can be an audio component of an audio/video feed (such as, for example, recording as a video cassette recorder (VCR) device would operate).

FIG. 1 shows an illustrative local operator LO, and an illustrative remote expert RE (e.g., supertech). However, in a Radiology Operations Command Center (ROCC) as contemplated herein, the ROCC provides a staff of supertechs who are available to assist local operators LO at different hospitals, radiology labs, or the like. Each remote expert RE can operate a corresponding remote workstation 12. The ROCC may be housed in a single physical location, or may be geographically distributed. For example, in one contemplated implementation, the remote expert RE are recruited from across the United States and/or internationally in order to provide a staff of supertechs with a wide range of expertise in various imaging modalities and in various imaging procedures targeting various imaged anatomies. A server computer 14s can be in communication with the medical imaging bay 3 and the remote service center 4 with one or more non-transitory storage media 26s. The non-transitory storage media 26s may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the server computer 14s, various combinations thereof, or so forth. It is to be understood that any reference to a non-transitory medium or media 26s herein is to be broadly construed as encompassing a single medium or multiple media of the same or different types. Likewise, the server computer 14s may be embodied as a single electronic processor or as two or more electronic processors. The non-transitory storage media 26s stores instructions executable by the server computer 14s.

The medical imaging device controller 10 in the medical imaging device bay 3 also includes similar components as the remote workstation 12 disposed in the remote service center 4. Except as otherwise indicated herein, features of the medical imaging device controller 10, which includes a local workstation 12′, disposed in the medical imaging device bay 3 similar to those of the remote workstation 12 disposed in the remote service center 4 have a common reference number followed by a “prime” symbol, and the description of the components of the medical imaging device controller 10 will not be repeated. In particular, the medical imaging device controller 10 is configured to display a GUI 28′ on a display device or controller display 24′ that presents information pertaining to the control of the medical imaging device 2, such as configuration displays for adjusting configuration settings an alert 30 perceptible at the remote location when the status information on the medical imaging examination satisfies an alert criterion of the imaging device 2, imaging acquisition monitoring information, presentation of acquired medical images, and so forth. It will be appreciated that the screen mirroring data stream 18 carries the content presented on the display device 24′ of the medical imaging device controller 10. The communication link 14 allows for screen sharing between the display device 24 in the remote service center 4 and the display device 24′ in the medical imaging device bay 3. The GUI 28′ includes one or more dialog screens, including, for example, an examination/scan selection dialog screen, a scan settings dialog screen, an acquisition monitoring dialog screen, among others. The GUI 28′ can be included in the video feed 17 and displayed on the remote workstation display 24 at the remote location 4.

Furthermore, as disclosed herein, the server 14s performs a method or process 100 for monitoring a medical imaging examination performed using a medical imaging device 2 (i.e., by assisting local operators LO of respective medical imaging devices 2 during medical imaging examinations by a remote expert RE). The instructions to perform the method 100 are stored in the non-transitory computer readable medium 26s of the server computer 14s.

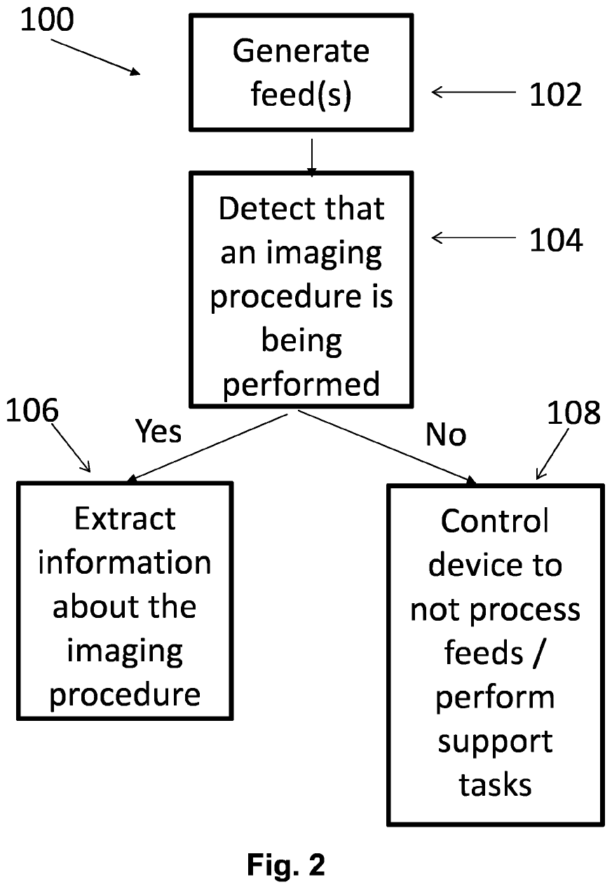

With reference to FIG. 2, and with continuing reference to FIG. 1, an illustrative embodiment of the method 100 in one aspect is diagrammatically shown as a flowchart. In this aspect, the edge devices 11 are leveraged to perform ML training tasks. To begin the method 100, an imaging examination is commenced by the local operator LO using the medical imaging device 2. An event can occur during the examination which requires assistance from a remote expert RE. At an operation 102, the video feed 17 (acquired by the one or more cameras 16 and/or the audio feed 18 (acquired by the one or more microphones 15) are routed to the server computer 14s for analysis. At an operation 104, the server computer 14s analyzes the video feeds 17 and/or the audio feeds 18 to detect whether a medical procedure is being performed in the imaging bay 3. For example, this can be based on whether the patient support is visible and does not have a patient loaded thereon. In another embodiment, the detection 104 can be based on information from the scraped imaging device controller 10, e.g. if an idle screen is detected then it is determined that the imaging device is not currently in use.

At an operation 106, in response to the detecting (i.e., the detecting operation 104) indicating a medical imaging procedure is being performed in the imaging bay 3, the server computer 14s is configured to control a local electronic processing device assigned to the imaging bay 3 (i.e., the ROCC device 8) to process the one or more video feeds 17 or audio feeds 18 to extract and present information about the medical procedure being performed in the imaging bay 3.

In another disclosed aspect of the operation 106, the one or more video feeds includes a video feed 17 comprising a scaped controller screen video feed of the medical imaging device controller 10 being used in the medical procedure being performed in the imaging bay 3. The controlling operation 106 can then include identifying text regions of the scraped controller screen video feed that contain text and categorizing the text regions as quasi-static or dynamic. An optical character recognition (OCR) process can be performed to extract content of the dynamic text regions continuously during the medical procedure being performed in the imaging bay 3, and another OCR process can be performed to extract content of the quasi-static text regions only at times of the medical procedure being performed in the imaging bay 3 at which content of the quasi-static text regions may change.

These are merely illustrative examples, and should not be construed as limiting.

At an operation 108, in response to the detecting indicating a medical procedure is not being performed in the imaging bay 3, the server computer 14s is configured to control the ROCC device 8 to not process the one or more video feeds 17 and/or audio feeds 18. For example, the controlling operation 108 can include controlling operation of the at least one camera 16 and/or the microphone 15 to not operate to prevent generation of the video feed 17 and/or the audio feed 18. Optionally, during these times when the ROCC device 11 is not being used for its primary purpose of supporting an imaging examination, it may be used for the secondary purpose of performing ROCC support tasks, such as training ML models used by the ROCC.

In some embodiments, the controlling operation 108 can include controlling the ROCC device 8 to apply at least one machine learning (ML) component 40 to extract information about medical imaging procedures performed in the imaging bay 3 from images acquired by the medical imaging device (e.g., to detect suboptimal imaging settings, to detect incidental findings, et cetera). For example, a plurality of ML models 40 can be retrieved from the server computer 14s, and training of at least one of the plurality of ML models 40 can be allocated to the ROCC device 8. In another example, when multiple ROCC devices 8 are provided in corresponding medical imaging device bays 3, each ROCC device 8 can be allocated one or more ML models 40 to train. In a further example, a first ML model 40 can be applied to extract a first type of information from the one or more video feeds 17 and/or audio feeds 18. Multiple second ML models 40 can be applied to extract a second type of information from the one or more video feeds 17 and/or audio feeds 18. The first and second types of information can then be combined (e.g., by a voting process) to extract the information presented about the medical procedure being performed in the imaging bay 3.

The results of the training of the ML models 40 can then be received at the server computer 14s from the ROCC device 8. The server computer 14s is then programmed to receive feedback from the ROCC device 8 indicative of performance of each ML model in extracting the information about the medical imaging procedures performed in the imaging bays 3. The server computer 14s is further programmed to allocate the ML model training tasks amongst the one or more ML models 40 based on the feedback received from the ROCC device(s) 8 indicative of performance of each ML model 40.

In some embodiments, the ROCC device 8 is configured to provide a communication interface (i.e., the natural language communication pathway 19) between the local operator LO and the remote expert RE.

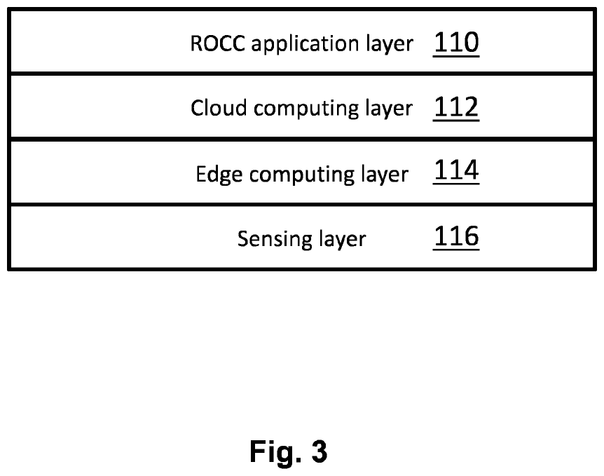

With reference to FIG. 3, a high level representation of the layered computational architecture of the ROCC is shown, including an ROCC application layer 110, a cloud computing layer 112, an edge computing layer 114, and a sensing layer 116. The bottom most layer is the sensing layer 116 which processes raw data from camera(s) 16 and the controller display. This pre-processed data from the sensing layer 116 is fed to ROCC tablet or other edge device 11 which is part of edge computing layer 114. Note that each scanner room or imaging bay in general has a dedicated ROCC tablet 11 containing a deep learning (DL), or other ML model (or suite of ML models) suited to its environment (specifically the scanner console and scanner room camera configuration). The edge computing layer 114 processes the data from the sensing layer 116 into events using the ML models. These events are streamed to the cloud account of the cloud computing layer 112 that is specific to the imaging center. The cloud computing layer 112 uses the events to measure workflow metrics and provide status on room, exam etc. This layer 112 also hosts a local repository of the ML models being used at a particular imaging center. The inferences from cloud computing layer 112 are pushed to the ROCC application layer 110 where it is relayed to expert users who can make appropriate imaging examination support decisions. The ROCC application layer 110 also contains the complete list of ML models used across the ROCC platform, to facilitate distributed training and update-training of the ML models.

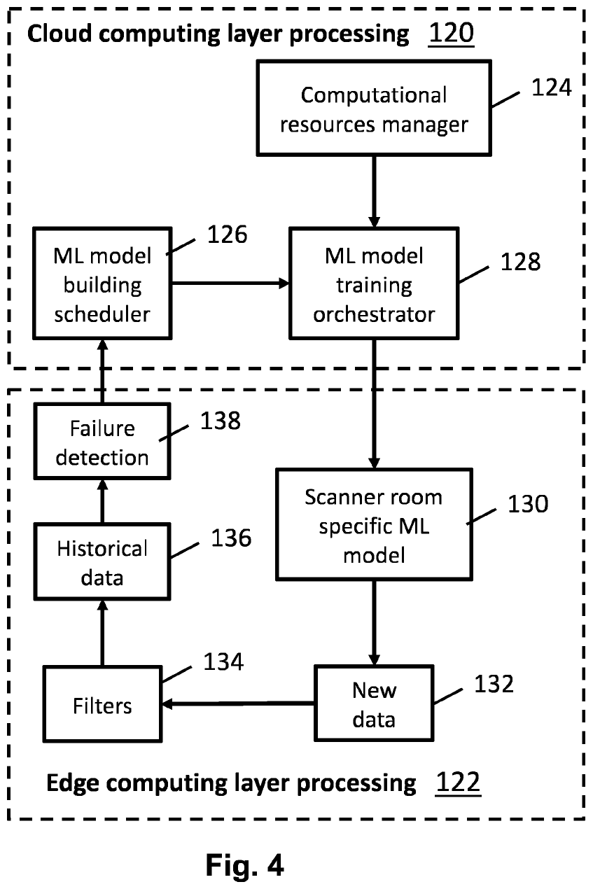

With reference to FIG. 4, an example is shown of leveraging the edge devices during operation 106 of FIG. 2 in accordance with the layered computational ROCC architecture of FIG. 3 to perform secondary ROCC support tasks, such as training or update-training of ML models used by the ROCC. The processing is divided between cloud computing layer processes 120 performed by the cloud computing layer 112, and processes 122 performed by the edge computing layer 114. The cloud computing processing 120 include a computational resources manager 124 manages the available computing resources. For example, it may receive the output 102 of the method 100 of FIG. 2 so as to determine which edge devices 11 are available for performing secondary support processing at any given time. A ML model building scheduler 126 schedules ML models to be trained, and a ML model training orchestrator assigns ML training tasks to edge devices based on their availability (from the resources manager 124) and the ML training tasks to be performed (from the scheduler 126). The tasks are then performed by the assigned edge devices per operation 106 of FIG. 2.

In the edge computing layer processing 122, at an operation 130 an edge device 11 uses a trained ML model to perform ROCC examination support tasks. For example, the operation 130 may use trained ML models to identify non-optimal imaging scan settings, incidental findings, or so forth in acquired images so as to provide early warning of such issues. The operation 130 may also use trained ML models applied to frames of the video feed(s) 17 to detect events occurring during the imaging examination. In applying the ML models, the edge device may use model weights, configuration parameters, or so forth that are specific to the imaging scanner 2 or camera 16 being used for the imaging examination. These applications of the ML models in operation 130 produce new data, which may optionally be filtered 134 in various ways to generate (new) historical data 136. A failure detection model 138 is applied to detect whether the ML model produced correct results. The operation 138 can operate in various ways, such as comparing the ML model output with information that confirms or contradicts that result. As an example, if the ML model outputs a warning that an imaging setting may be incorrect, and the technician adjusts that setting and reacquires the data then this is confirmatory; if on the other hand the technician saves the files without rescanning this is contradictory. The operation 138 thus provides “ground truth” labels as to whether the ML model output is correct or incorrect. Optionally, this feedback may be collected by providing the imaging technician with a short feedback dialog, e.g. asking: “Was this information helpful?” The labeled historical data are then fed back to the ML model building scheduler 126 to perform model update training as appropriate. (For example, if a model has more than some threshold percent contradiction it may be update-trained).

### EXAMPLE

The following describes the support apparatus 1 in more detail. The server computer 14s can receive data from a sensing layer configured to process the video feeds 17 and the audio feeds 18. This pre-processed data can be input to the ROCC device 8 (comprising an edge computing layer) that can include one of the ML models 40 configured to process events during the medical imaging examination. The processed data are transmitted to the server computer 14s and analyzed to measure workflow metrics and provide status updates on the medical imaging examination. The server computer 14s also stores the ML Models 40.

The ML models can include a You Only Look Once (YOLO) or another object detection algorithm to generate an event stream. To generate a reliable and accurate event stream, these models 40 need to be trained regularly. The training of these models takes about 200 hours over a dataset containing 5000 images on the ROCC device 8. In addition, the number of ML models 40 run on ROCC devices 8 is directly proportional to the number of variants in the imaging bay 3. Given this setup, the training is a repetitive process involving intense computational activities.

The computational resources available in the edge computing layer can be used to perform different services such as ML model 40 training, convert sensed data into events, etc. A computational resource manager tracks the services (e.g., sensing, training, etc.) offered by each resource. The console and camera pipelines are triggered OFF when there is no activity on the console screen and scanner room, respectively. This usually occurs when there is a long-time gap between two consecutive exams and night shifts. In these time periods, the services rendered by the computational sources on the network of ROCC devices 8 are switched to training the ML models 40. This information is provided to a training orchestrator module implemented in the server computer 14s.

The training orchestrator module which takes inputs from a ML model building scheduling module and a computational resource manager implemented in the server computer 14s. The ML model building scheduling module identifies the ML models 40 that require training whereas the computational resource manager identifies the available computational resources within the network of ROCC devices 8 installed at an imaging center. These inputs are used by training orchestrator module to execute the training of the ML models 40. However, to initiate the training of a ML model 40, the training orchestrator module compares the current ML model 40 located in a local model repository implemented in the ROCC device 8 with the corresponding ML model 40 in the global model repository implemented in the server computer 14s. The training is initiated only if both the ML models 40 are identical. For performance improvements, the training job of a ML model 40 is scheduled based on current and target performance metrics (e.g. the number of failures seen in the results obtained from the current deployed ML model 40 and the time since the ML model 40 is last trained). Once the training is complete, the old ML model 40 is replaced with the new trained ML model 40 in both local and global DL model repositories. The computational resource manager chooses the available resources in a way that it balances the workload among the computational resources while reducing the wait time for execution of tasks that are in the queue. To achieve this, it deploys optimization models such as a gradient descent, genetic algorithm. Workload balancing requires pausing ML model training, saving/loading current training states, and restarting ML training without compromising the performance of any tablet to provide the high-priority services e.g. audio-video telepresence, execution of the console and camera algorithms once activity is detected on those input channels, etc.

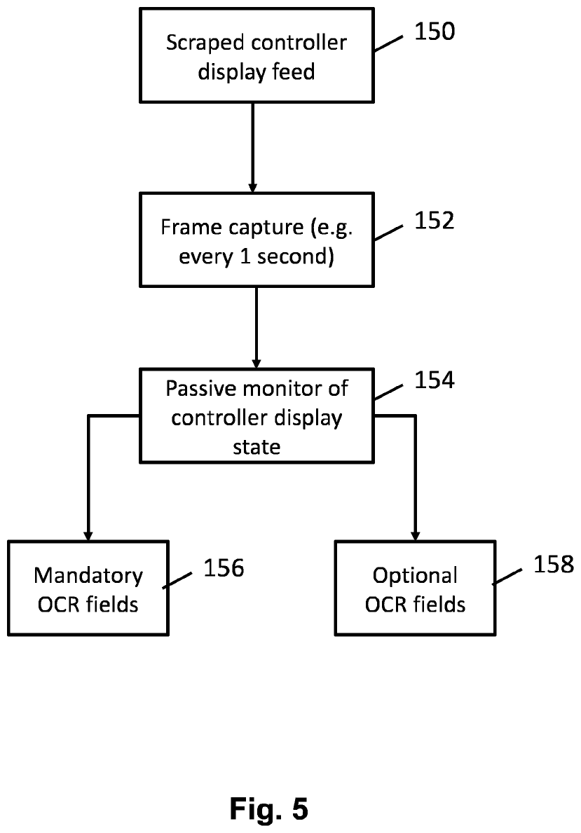

With reference to FIG. 5, in an aspect of the operation 106 of FIG. 2 the OCR of text of the scraped or controller display 24′ may be limited based on content. To this end, the process receives the scraped controller display feed 150, and captures a frame for processing in an operation 152 at, for example, one frame captured per second. (The capture rate is chosen to balance processing load which is reduced by using a lower capture frequency versus time resolution or responsiveness is increased by using a higher capture frequency). In an operation 154, the display is monitored to detect and classify text fields. This can for example be done by applying a priori known controller display templates, such as templates for the patient intake display, the scan setup display, the acquisition monitoring display, the image review display, and/or so forth. These templates may be specific to the make/model of the medical imaging device. Additionally or alternatively, the current frame can be compared with the last-captured frame to detect changes. Each text region is then classified as a mandatory OCR field 156 or as an optional OCR field 158. As a specific example, an ROCC console event stream is generated by OCR and passive monitor pipelines. An OCR pipeline is run on multiple fields (>10) on the console screen of the medical imaging device controller 10. It takes about 1-2 seconds per field to execute the OCR pipeline. This execution may be performed sequentially, and the time taken by OCR pipeline puts a limit on the sensing frequency from the console feed. To address this, the server computer 14s applies the process of FIG. 4 and thereby classifies all the fields into two categories (Mandatory and Optional) and use OCR on both categories via parallel processing to reduce the computational time. Optional fields are those fields that undergo changes during an exam start but remain same throughout the exam. Running OCR on these fields are conditioned on certain events such as exam start. The remaining fields fall under Mandatory category. Techniques such as multiprocessing, multithreading are used to run OCR in parallel on mandatory and optional fields. To further reduce the computational burden, the passive monitor 154 is introduced. The passive monitor 154 uses fast and simple image analysis (e.g., template matching or comparison with the last-captured frame) to determine if a pop-up window is present in the image or if there is any change in the current image from previous image. The passive monitor triggers relevant OCR pipeline (Mandatory 156 or optional 158 or both, i.e. for different fields of a single captured controller display frame) based on its findings, so that the OCR pipeline is not run unnecessarily.

A YOLO ML model 40 is used to detect a region of interest (ROI) for a given OCR field in an image. Variations in the text background and a font size of these fields translate in the ROI detection by the YOLO model. It is very important to identify the ROI for each OCR field correctly. The current implementation relies on a single YOLO model making it prone to misdetections. To address this, multiple YOLO models (i.e., five) can be used to detect the fields on a given image. Consensus on the detection of the field is derived by using mechanisms such as a voting process.

Unlike a single model implementation, the best ROI is identified by multiple ML models 40. Different merging strategies such as overlapping ROI or F1-score weighted combination of ROIs can be used to determine the best ROI. This way of using ensemble of the ML models 40 to determine the bounding box makes the ROI detection robust. Running multiple ML models 40 on an image require additional computation time. To overcome this, these ML models 40 are run using parallel processing and trained using a service switching process.

The computer system may also include a processor. The processor executes instructions to implement some or all aspects of methods and processes described herein. The processor is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor is an article of manufacture and/or a machine component. The processor is configured to execute software instructions to perform functions as described in the various embodiments herein. The processor may be a general-purpose processor or may be part of an application specific integrated circuit (ASIC). The processor may also be a microprocessor, a microcomputer, a processor chip, a controller, a microcontroller, a digital signal processor (DSP), a state machine, or a programmable logic device. The processor may also be a logical circuit, including a programmable gate array (PGA), such as a field programmable gate array (FPGA), or another type of circuit that includes discrete gate and/or transistor logic. The processor may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system further includes a main memory and a static memory, where memories in the computer system communicate with each other and the processor via a bus. Either or both of the main memory and the static memory may be considered representative examples of the memory of the controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory and the static memory are articles of manufacture and/or machine components. The main memory and the static memory are computer-readable mediums from which data and executable software instructions can be read by a computer (e.g., the processor). Each of the main memory and the static memory may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, Blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system further includes a video display unit, such as a liquid crystal display (LCD), an organic light emitting diode (OLED), a flat panel display, a solid-state display, or a cathode ray tube (CRT), for example. Additionally, the computer system includes an input device, such as a keyboard/virtual keyboard or touch-sensitive input screen or speech input with speech recognition, and a cursor control device, such as a mouse or touch-sensitive input screen or pad. The computer system also optionally includes a disk drive unit, a signal generation device, such as a speaker or remote control, and/or a network interface device.

In an embodiment, the disk drive unit includes a computer-readable medium in which one or more sets of software instructions are embedded. The sets of software instructions are read from the computer-readable medium to be executed by the processor. Further, the software instructions, when executed by the processor, perform one or more steps ofthe methods and processes as described herein. In an embodiment, the software instructions reside all or in part within the main memory, the static memory and/or the processor during execution by the computer system. Further, the computer-readable medium may include software instructions or receive and execute software instructions responsive to a propagated signal, so that a device connected to a network communicates voice, video or data over the network. The software instructions may be transmitted or received over the network via the network interface device.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true scope of the present disclosure. Thus, to the maximum extent allowed by law, the scope of the present disclosure is to be determined by the broadest permissible interpretation of the following claims and their equivalents and shall not be restricted or limited by the foregoing detailed description.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A method of monitoring a medical imaging examination, the method comprising the steps of:
receiving one or more video feeds of at least an imaging bay;
detecting, from the one or more video feeds, whether a medical procedure is being performed in the imaging bay;
in response to the detecting indicating a medical procedure is being performed in the imaging bay, controlling a local electronic processing device assigned to the imaging bay to process the one or more video feeds to extract and present information about the medical procedure being performed in the imaging bay; and
in response to the detecting indicating a medical procedure is not being performed in the imaging bay, controlling the local electronic processing device to not process the one or more video feeds.

2. The method of claim 1 further including the steps of:
receiving an audio feed acquired by at least one microphone disposed in the imaging bay;
wherein the controlling of the electronic processing device to not process the one or more video feeds further includes controlling operation of the at least one camera or the microphone to not operate to prevent generation of the video feed and the audio feed.

3. The method of claim 1, wherein the method further includes:
in response to the detecting indicating a medical procedure is not being performed in the imaging bay, controlling the local electronic processing device to perform one or more training tasks for a machine learning (ML) component.

4. The method of claim 3, wherein the method further includes:
retrieving a plurality of ML models; and
allocating training of at least one of the plurality of ML models to the local electronic processing device.

5. The method of claim 1, wherein the one or more video feeds includes a video feed (17) comprising a scraped controller screen video feed of a medical imaging device controller being used in the medical procedure being performed in the imaging bay, and the controlling of the local electronic processing device to process the one or more video feeds to extract and present information about the medical procedure being performed in the imaging bay includes:
identifying text regions of the scraped controller screen video feed that contain text; and
categorizing the text regions as quasi-static or dynamic;
performing optical character recognition to extract content of the dynamic text regions continuously during the medical procedure being performed in the imaging bay; and
performing OCR to extract content of the quasi-static text regions only at times of the medical procedure being performed in the imaging bay at which content of the quasi-static text regions may change.

6. The method of claim 1, wherein the controlling of the local electronic processing device to process the one or more video feeds to extract and present information about the medical procedure being performed in the imaging bay includes:
applying a first machine-learning model to extract first information from the one or more video feeds;
apply a plurality of second ML models to extract second information from the one or more video feeds; and
combining the first and second information to extract the information presented about the medical procedure being performed in the imaging bay.

7. The method of claim 6, wherein the combining the first and second information comprises using a voting process.

8. The method of claim 1, wherein the local electronic processing device is further programmed to provide a communication interface between a user of the local electronic processing device and a remote expert located remotely from the imaging bay to which the local electronic processing device is assigned.

9. A support apparatus for medical imaging, the support apparatus comprising:
a server computer; and
local electronic processing devices assigned to respective medical imaging bays (3) and programmed to apply machine learning models to video feeds received from their respective assigned imaging bays to extract information about medical imaging procedures performed in their respective assigned imaging bays;
wherein the server computer and/or the local electronic processing devices are programmed to determine whether medical imaging procedures are being performed in the respective medical imaging bays; and
wherein the server computer is programmed to perform training of the ML models including allocating ML model training tasks amongst the local electronic processing devices based on whether medical imaging procedures are being performed in the corresponding assigned medical imaging bays and receiving results of the allocated ML model training tasks from the local electronic processing devices.

10. The support apparatus of claim 9, wherein:
the server computer receives feedback from the local electronic processing devices indicative of performance of each ML model of the ML models in extracting the information about the medical imaging procedures performed in the respective assigned imaging bays; and
the server computer is further programmed to allocate the ML model training tasks amongst the ML models based on the feedback received from the local electronic processing devices indicative of performance of each ML model.

11. The support apparatus of claim 9, wherein each local electronic processing device is further programmed to provide a communication interface between a user of the local electronic processing device and a remote expert located remotely from the imaging bay to which the local electronic processing device is assigned.

12. A non-transitory computer readable medium storing instructions executable by at least one electronic processing device to perform a method of monitoring a medical imaging examination, the method comprising:
receiving one or more video feeds of at least an imaging bay;
detecting, from the one or more video feeds, whether a medical procedure is being performed in the imaging bay;
in response to the detecting indicating a medical procedure is being performed in the imaging bay, controlling a local electronic processing device assigned to the imaging bay to process the one or more video feeds to extract and present information about the medical procedure being performed in the imaging bay; and
in response to the detecting indicating a medical procedure is not being performed in the imaging bay, controlling the local electronic processing device to perform one or more training tasks for a machine learning component.

13. The non-transitory computer readable medium of claim 12, wherein the non-transitory computer readable medium further includes carrying out the steps of:
receiving an audio feed acquired by at least one microphone disposed in the imaging bay;
wherein the controlling of the electronic processing device to not process the one or more video feeds further includes controlling operation of the at least one camera or the microphone to not operate to prevent generation of the video feed and the audio feed.

14. The non-transitory computer readable medium of claim 12, wherein the non-transitory computer readable medium further includes carrying out the steps of:
in response to the detecting indicating a medical procedure is not being performed in the imaging bay, controlling the local electronic processing device to not process the one or more video feeds.

15. The non-transitory computer readable medium of claim 12, wherein the method further includes:
retrieving a plurality of ML models; and
allocating training of at least one of the plurality of ML models to the local electronic processing device.

16. A computer program comprising instructions which, when the program is executed by a computer, cause the computer to carry out the steps of claim 1.
